(19) United States
(12) Patent Application Publication
(10) Pub. No.: US 2022/0054397 A1
(43) Pub. Date: Feb. 24, 2022

(54) Compositions for Treatment of Nails and skin

(71) Applicant: Adriana Strimbu, Hallandale Beach, FL (US)

(72) Inventor: Adriana Strimbu, Hallandale Beach, FL (US)

(21) Appl. No.: 17/405,280

(22) Filed: Aug. 18, 2021

Related U.S. Application Data
(60) Provisional application No. 63/067,069, filed on Aug. 18, 2020.

(51) Int. Cl. A61K 8/9789; A61K 8/92; A61K 8/67; A61K 8/46; A61K 8/36; A61Q 19/10; A61Q 3/00

(52) U.S. Cl. CPC A61K 8/9789; A61K 8/922; A61K 8/678; A61Q 3/00; A61K 8/361; A61Q 19/10; A61K 8/46

(57) ABSTRACT

The present invention is a topical composition for treating nail maladies comprising synergistic ratios of Castor Oil, Vitamin E Oil, Eucalyptus Oil, Lavender Oil, Tea Tree Oil, and Thyme Oil.

### INDEX TO RELATED APPLICATIONS

This application is a non-provisional of and claims benefit to U.S. Provisional Patent Application Ser. No. 63/067,069 filed Aug. 18, 2020, the disclosure of which is incorporated herein by reference in its entirety.

### BACKGROUND OF THE INVENTION

Persons experiencing anomalies with their fingernails and toenails often encounter difficulty in obtaining affective treatment. There are many pharmaceutical and non-pharmaceutical preparations available to treat such conditions. As is often the case, persons experience varying degrees of improvement.

Medical practitioners have been researching for years in order to formulate a composition that provides improvement in a very significant percentage of affective persons.

The present invention has achieved this goal.

### SUMMARY OF THE INVENTION

While various components used in the formulation of the present invention have been used in topical compositions, the present invention has discovered a particular ratio exhibiting a unique synergistic affect in the treatment of nail maladies. The invention has further been discovered to be a useful component as a foot soak. It cannot be emphasized enough that the present invention is not merely a collection of components. A critical feature of the present invention is the ratio in which the components are used. The critical ratios provide synergistic effects that have not been seen until now.

In one embodiment the ratio is:
Castor Oil 8-9.6: Vitamin E Oil 0.15-0.25: Eucalyptus Oil 0.15-0.25: Lavender Oil 0.15-0.25: Tea Tree Oil10.15-0.25: Thyme Oil 0.15-0.25.

In a preferred embodiment the ratio is
Castor Oil 9: Vitamin E Oil 0.2: Eucalyptus Oil 0.2: Lavender Oil 0.2: Tea Tree Oil 0.2: Thyme Oil 0.2.

Another embodiment has the ratios
Castor Oil 9.6: Vitamin E Oil 0.2: Eucalyptus Oil 0.05: Lavender Oil 0.05: Tea Tree Oil 0.05: Thyme Oil 0.05.

In one embodiment, the invention is a composition for treating human nails comprising:
the synergistic ratio of Castor Oil 8-9.6: Vitamin E Oil 0.15-0.25: Eucalyptus Oil 0.15-0.25: Lavender Oil 0.15-0.25: Tea Tree Oil10.15-0.25: Thyme Oil 0.15-0.25.

In one embodiment, the composition is formed with a synergistic ratio of components comprises: Castor Oil 9: Vitamin E Oil 0.2: Eucalyptus Oil 0.2: Lavender Oil 0.2: Tea Tree Oil 0.2: Thyme Oil 0.2.

In one embodiment, the composition is formed with a synergistic ratio of components comprises: Castor Oil 9.6: Vitamin E Oil 0.2: Eucalyptus Oil 0.05: Lavender Oil 0.05: Tea Tree Oil 0.05: Thyme Oil 0.05.

In one embodiment, the Vitamin E Oil is DL-Alpha-Tocopheryl Acetate CAS No. 7695-91-2.

In one embodiment, the Eucalyptus Oil is Eucalyptus Oil having CAS 8000-48-4.

In one embodiment, the Lavender Oil is Pure Lavandin (Abrialis) Essential Oil with the Botanical Name, Lavandula intermedia var Abrialis.

In one embodiment, the Tea Tree Oil is Melaleuca quinquenervia oil having CAS 68647-73-4.

In one embodiment, the compositions disclosed herein are incorporated into a viscous cream or gel for topical administration on nails, feet, skin, or combinations thereof.

In one embodiment, the Tolnaftate solution, Undecylenic acid, or combinations thereof.

In one embodiment, the invention is a composition for treating human nails consisting of:
the synergistic ratio of Castor Oil 8-9.6: Vitamin E Oil 0.15-0.25: Eucalyptus Oil 0.15-0.25: Lavender Oil 0.15-0.25: Tea Tree Oil0.15-0.25: Thyme Oil 0.15-0.25.

In one embodiment, the composition is formed with a synergistic ratio of components consisting of: Castor Oil 9: Vitamin E Oil 0.2: Eucalyptus Oil 0.2: Lavender Oil 0.2: Tea Tree Oil 0.2: Thyme Oil 0.2.

In one embodiment, the composition is formed with a synergistic ratio of components consisting of: Castor Oil 9.6: Vitamin E Oil 0.2: Eucalyptus Oil 0.05: Lavender Oil 0.05: Tea Tree Oil 0.05: Thyme Oil 0.05.

### DETAILED DESCRIPTION OF THE PREFERRED EMBODIMENTS

The present invention provides a unique combination in a synergistic beneficial ratio to achieve the desired treatment of nail maladies

The base is Castor Oil.

A preferred Castor Oil includes Ricinoleic Acid, Oleic Acid, Linoleic Acid (Omega-6 Fatty Acid), α-Linolenic Acid (Alpha-Linolenic Acid-Omega-3 Fatty Acid), Stearic Acid, and Palmitic Acid.

A preferred eucalyptus is CAS 8000-48-4.

A preferred lavender is Pure Lavandin (Abrialis) Essential Oil with the Botanical Name: Lavandula intermedia var Abrialis.

A preferred Tea Tree Oil, also known as Melaleuca quinquenervia oil has CAS 68647-73-4.

Thyme Certified Organic Essential Oil is preferred.

Toenail Fungus (Onychomycosis)as is known includes main organisms of the toenail fungus are Trichophyton Rubrum and Trichophyton mentagrophytes.

Some other organisms commonly found in toenail fungus are Candida albicans, Aspergillus species.

If someone seeks medical attention to get treated for toenail fungus topical prescription antifungal medication or oral antifungal medication are often prescribed.

Topical antifungal medication such as Ciclopirox 8% solution, Jublia 10% solution, Kerydin solution, or other creams and topicals that are used off label for toenail fungus like: Ciclopirox 0.77% gel, Naftin cream/gel, Ketoconazole cream.

The oral antifungal medication is usually reserved for patients that do not respond to topical antifungal medications and many of the patients cannot go on oral medication due to their health problems. Others do not want to go on oral antifungal medication even if they do not have any health issues.

Many patients do not want oral antifungal treatment because they are afraid of the side effects, they can have such as liver disease, etc.

Many of the patients then will request to use topical antifungal treatment.

Patients with systemic diseases, like diabetes, neuropathy, peripheral vascular disease are at risk and require foot care on regular basis.

The present invention has been observed in a clinical setting as being beneficial and exhibiting improvement in at least:
Patients that who have dry, brittle nails and want to restore their nails to normal consistency;
Patients who have dry, hard cuticles and want to soften them;
Patients who have fungus nails, yellow, hard, thick toenails and are looking to give the nail that soft natural look of the nail;
Patients who have hard, thick toenails, have been to the doctor and have been told they do not have fungus;
Patients with damaged nails due to psoriasis or trauma;
Patients who just want to maintain clean and healthy nails and cuticles.

It is further contemplated the present invention be applied alone, or in combination with any one or more of

Tolnaftate solution;

Undecylenic acid;

Tolnaftate solution+Undecylenic solution.

It is further contemplated the composition of the present invention be incorporated into a cream or gel to make it an antifungal cream or gel either alone, or with the aforementioned Tolnaftate solution;

Undecylenic acid; and/or

Tolnaftate solution+Undecylenic solution.

In one embodiment, the present invention is incorporated into a foot soak solution as follows:

8 ml of the any of the compositions disclosed herein are combined with a liquid suspension soap bas suitable for use in foot baths.

As understood from this disclosure, 8 ml of the composition being:
components exhibiting a synergistic ratio of Castor Oil 8-9.6: Vitamin E Oil 0.15-0.25: Eucalyptus Oil 0.15-0.25: Lavender Oil 0.15-0.25: Tea Tree Oil0.15-0.25: Thyme Oil 0.15-0.25
as well as any one of the compositions having ratios disclosed herein.

The present invention contemplates combination with any commercially available product or product similarly formulated being a dermatologically acceptable liquid soap base.

In one embodiment, the soap base is Cratfer'sChoice® Liquid Suspension Soap Base.

The aforementioned 8 ml of the composition according to the present invention is combined with 115-125 ml of the liquid soap base.

After combining, 10 ml of the combined solution is added to one gallon of water and used as a foot soak.

While the invention has been described in its preferred form or embodiment with some degree of particularity, it is understood that this description has been given only by way of example and that numerous changes in the details of construction, fabrication, and use, including the combination and arrangement of parts, may be made without departing from the spirit and scope of the invention.

I claim:

1. A composition for treating human nails comprising:
a combination of components exhibiting a synergistic ratio, said components and ratios are: Castor Oil 8-9.6: Vitamin E Oil 0.15-0.25: Eucalyptus Oil 0.15-0.25: Lavender Oil 0.15-0.25: Tea Tree Oil0.15-0.25: Thyme Oil 0.15-0.25.

2. The composition of claim 1 wherein the synergistic ratio of components comprises: Castor Oil 9: Vitamin E Oil 0.2: Eucalyptus Oil 0.2: Lavender Oil 0.2: Tea Tree Oil 0.2: Thyme Oil 0.2.

3. The composition of claim 1 wherein the synergistic ratio of components comprises: Castor Oil 9.6: Vitamin E Oil 0. 2: Eucalyptus Oil 0.05: Lavender Oil 0.05: Tea Tree Oil 0.05: Thyme Oil 0.05.

4. The composition of claim 1 wherein said Eucalyptus Oil is Eucalyptus Oil having CAS 8000-48-4.

5. The composition of claim 1 wherein said Lavender Oil is Pure Lavandin (Abrialis) Essential Oil with the Botanical Name, Lavandula intermedia var Abrialis.

6. The composition of claim 1 wherein said Tea Tree Oil is Melaleuca quinquenervia oil having CAS 68647-73-4.

7. The composition of claim 1 incorporated into a viscous cream or gel.

8. The composition of claim 1 further comprising Tolnaftate solution, Undecylenic acid, or combinations thereof.

9. A composition for treating human nails consisting of: components exhibiting a synergistic ratio of Castor Oil 8-9.6: Vitamin E Oil 0.15-0.25: Eucalyptus Oil 0.15-0.25: Lavender Oil 0.15-0.25: Tea Tree Oil0.15-0.25: Thyme Oil 0.15-0.25.

10. The composition of claim 9 wherein the synergistic ratio of components consists of: Castor Oil 9: Vitamin E Oil 0.2: Eucalyptus Oil 0.2: Lavender Oil 0.2: Tea Tree Oil 0.2: Thyme Oil 0.2.

11. The composition of claim 9 wherein the synergistic ratio of components consists of: Castor Oil 9.6: Vitamin E Oil 0. 2: Eucalyptus Oil 0.05: Lavender Oil 0.05: Tea Tree Oil 0.05: Thyme Oil 0.05.

12. A foot soak composition, comprising: 8 ml of a combination of components exhibiting a synergistic ratio, said components and ratios are: Castor Oil 8-9.6: Vitamin E Oil 0.15-0.25: Eucalyptus Oil 0.15-0.25: Lavender Oil 0.15-0.25: Tea Tree Oil10.15-0.25: Thyme Oil 0.15-0.25, combined with 115-125 ml of a dermatologically acceptable liquid soap base to form a primary solution;
adding 10 ml of said primary solution to one gallon of water to form said foot soak composition.

13. The composition of claim 12 wherein the synergistic ratio of components comprises: Castor Oil 9: Vitamin E Oil 0.2: Eucalyptus Oil 0.2: Lavender Oil 0.2: Tea Tree Oil 0.2: Thyme Oil 0.2.

14. The composition of claim 12 wherein the synergistic ratio of components comprises: Castor Oil 9.6: Vitamin E Oil 0.2: Eucalyptus Oil 0.05: Lavender Oil 0.05: Tea Tree Oil 0.05: Thyme Oil 0.05.
